(19) United States
(12) Patent Application Publication
(10) Pub. No.: US 2013/0165520 A1
(43) Pub. Date: Jun. 27, 2013

(54) Hybrid Molecule Having Mixed Retinoic Acid Receptor Agonism and Histone Deacetylase Inhibitory Properties

(71) Applicants: Wilson H. Miller, Montreal (CA); Sylvie Mader, Montreal (CA); James Gleason, Montreal (CA)

(72) Inventors: Wilson H. Miller, Montreal (CA); Sylvie Mader, Montreal (CA); James Gleason, Montreal (CA)

(73) Assignees: UNIVERSITY DE MONTREAL, Montreal, QC (CA); THE ROYAL INSTITUTE FOR THE ADVANCEMENT OF LEARNING/MCGILL UNIVERSITY, Montreal, QC (CA)

(21) Appl. No.: 13/578,057

(22) PCT Filed: Feb. 11, 2011
(86) PCT No.: PCT/CA2011/000166

§ 371 Date: Oct. 15, 2012

Related U.S. Application Data
(60) Provisional application No. 61/303,545, filed on Feb. 11, 2010.

(51) Int. Cl. C07C 259/10

(52) U.S. Cl. CPC C07C 259/10
USPC 514/575; 562/622; 435/375

(57) ABSTRACT

Hybrid molecules comprising a retinoic acid receptor agonist moiety and a histone deacetylase inhibitor (HDAC) moiety are disclosed. Hybrid molecule 3 (6-(5,5,8,8-tetramethyl-6,7-dihydronaphthalen-2-yl)naphthalene-2-hydroxamic acid) was proven to posses HDAC activity while maintaining RAR agonist activity. Hybrid molecule 3 and pharmaceutical compositions thereof can be used in the treatment of breast cancer, leukemia, non-small cell lung cancer, colon cancer, melanoma, ovarian cancer, renal cancer, prostate cancer and cancer of the CNS.

This application claims priority to U.S. Provisional Application No. 61/303,545 filed on Feb. 11, 2010, the entire disclosures of which are specifically incorporated herein by reference in their entirety without disclaimer.

### FIELD

The present disclosure relates to a new chemical agent that demonstrates antiproliferative and cytotoxic activity against cancer cells. More particularly, but not exclusively, the present disclosure relates to a hybrid molecule capable of mixed retinoic acid receptor agonism and histone deacetylase inhibition. The present disclosure also relates to methods of synthesis.

### BACKGROUND

Both retinoids and histone deacetylase inhibitors (HDACi) have been shown to possess anti-tumor properties in the clinic and have been shown to work cooperatively in combination in pre-clinical models. Retinoids can inhibit the growth of normal mammary epithelial cells and breast cancer cell lines by inducing G1 arrest and/or apoptosis.^{[1-6]} However, despite their promise in breast cancer cell lines, retinoids have not performed well in breast cancer treatment, although they were shown to reduce second malignancies in the breast.^{[7-9]} This may be due to intrinsic and/or acquired resistance, which can readily be observed in vitro in breast cancer and leukemic cell lines.^{[10-13]} While estrogen receptor (ER) positive (ER+ve) cells, such as MCF7, are sensitive to the anti-proliferative effects of ATRA (Vesanoid®), most ER negative (ER-ve) cells are not.^{[14]} This may be due to induction of RARα expression by estrogens and/or to other levels of cross-talk between the two receptors.^{[15-20]} HER2 amplification, which occurs in 25% of breast tumors, has been reported to correlate with lack of ERα expression and resistance to RA.^{[}^{21]}

HDACi's have shown promise in pre-clinical models of solid tumors including breast cancer. It was shown that HDACi's repress transcription of ERα, a therapeutic target for ⅔ of breast tumors.^{[22]} In addition, HDACi's down-regulate HER2, a proto-oncogene amplified in 25% breast tumors, both at the transcriptional level and through increased HER2 protein turnover, and sensitize HER2 amplified breast cancer cells such as SkBr3 to herceptin or chemotherapeutic drug treatment. ^{[23,24]}

It was previously reported that HDACi's synergize with RA (retinoic acid) to inhibit growth and induce apoptosis in breast cancer cells.^{[25]} While both RA and the HDACi trichostatin A are anti-proliferative in MCF7 cells, comparison of patterns of gene expression upon treatment with either compound revealed only partial overlap in regulated genes, possibly explaining their cooperative action; e.g., while RA and TSA induce expression of CDKI p19,^{[26,27]} TSA but not RA strongly suppressed expression of cyclin CCND1. Previous reports have shown that the combination of retinoic acid receptor agonists with histone deacetylase inhibitors can be advantageous in leukemia and breast cancer.

Triciferol, a hybrid molecule which combines vitamin D receptor agonism with HDACi activity and which displays improved cytostatic and cytotoxic activity relative to 1,25-dihydroxyvitamin D was previously reported.

The present description refers to a number of documents, the content of which is herein incorporated by reference in their entirety.

### SUMMARY

The present disclosure relates to a hybrid molecule capable of mixed retinoic acid receptor agonism and histone deacetylase inhibition.

In an embodiment, the present disclosure relates to a hybrid molecule comprising a retinoic acid receptor agonist moiety and an HDAC inhibitor moiety.

In an embodiment, the present disclosure relates to a hybrid molecule or a pharmaceutically acceptable salt thereof having the formula:

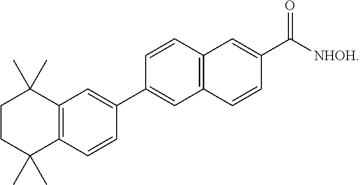

In an embodiment, the present disclosure relates to a pharmaceutical composition comprising an effective amount of the hybrid molecule or a pharmaceutically acceptable salt thereof having the formula:

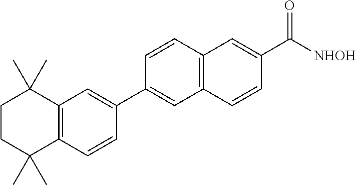

in association with one or more pharmaceutically acceptable carriers, excipients or diluents.

In an embodiment, the present disclosure relates to an admixture comprising an effective amount of the hybrid molecule or a pharmaceutically acceptable salt thereof having the formula:

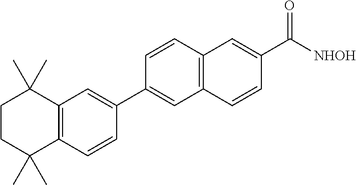

in association with one or more pharmaceutically acceptable carriers, excipients or diluents.

In an embodiment, the present disclosure relates to a method of treating breast cancer in a subject comprising administering to the subject a therapeutically effective amount of hybrid 3 or a pharmaceutically acceptable salt thereof.

In an embodiment, the present disclosure relates to a method of treating leukemia in a subject comprising administering to the subject a therapeutically effective amount of hybrid 3 or a pharmaceutically acceptable salt thereof.

In an embodiment, the present disclosure relates to a method of treating non-small cell lung cancer in a subject comprising administering to the subject a therapeutically effective amount of hybrid 3 or a pharmaceutically acceptable salt thereof.

In an embodiment, the present disclosure relates to a method of treating colon cancer in a subject comprising administering to the subject a therapeutically effective amount of hybrid 3 or a pharmaceutically acceptable salt thereof.

In an embodiment, the present disclosure relates to a method of treating melanoma in a subject comprising administering to the subject a therapeutically effective amount of hybrid 3 or a pharmaceutically acceptable salt thereof.

In an embodiment, the present disclosure relates to a method of treating ovarian cancer in a subject comprising administering to the subject a therapeutically effective amount of hybrid 3 or a pharmaceutically acceptable salt thereof.

In an embodiment, the present disclosure relates to a method of treating renal cancer in a subject comprising administering to the subject a therapeutically effective amount of hybrid 3 or a pharmaceutically acceptable salt thereof.

In an embodiment, the present disclosure relates to a method of treating prostate cancer in a subject comprising administering to the subject a therapeutically effective amount of hybrid 3 or a pharmaceutically acceptable salt thereof.

In an embodiment, the present disclosure relates to a method of treating cancer of the central nervous system in a subject comprising administering to the subject a therapeutically effective amount of hybrid 3 or a pharmaceutically acceptable salt thereof.

In an embodiment of the present disclosure, the subject to be treated is an in vitro or in vivo system. In a further embodiment of the present disclosure, the subject is a human.

The foregoing and other objects, advantages and features of the present disclosure will become more apparent upon reading of the following non restrictive description of illustrative embodiments thereof, given by way of example only with reference to the accompanying drawings.

### BRIEF DESCRIPTION OF THE DRAWINGS

In the appended drawings:

FIG. 1 shows the virtual docking of TTNN (left) and hybrid 3 (right) to RARγ.

FIG. 2a shows the effects of hybrid 3, ATRA (1), TTNN (2) and HX600 on RAR ligand binding using the BRET assay; FIG. 2b shows the effects of TTNN and hybrid 3 in inducing RARα.

FIG. 3 shows the effects of hybrid 3 on either RAR or HDAC target gene regulation in the MDA-MB-231 and MCF7 cells lines.

FIG. 4 shows the hyperacetylation of proteins resulting from the effects of hybrid 3 on protein deacetylases.

FIG. 5 shows the effects of hybrid 3, TTNN, ATRA and SAHA on the growth of the MDA-MB-231, MCF7 and SkBr3 cell lines.

### DETAILED DESCRIPTION

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this disclosure pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The present description refers to a number of chemical terms and abbreviations used by those skilled in the art. Nevertheless, definitions of selected terms are provided for clarity and consistency.

A novel class of chemical agents (i.e. a novel hybrid molecule) having mixed retinoic acid receptor agonism and histone deacetylase inhibitory properties are described herein.

The incorporation of histone deacetylase inhibitory activity (HDACi) into an RAR agonist results in a molecule with improved antiproliferative activity towards several breast cancer cell lines, including MCF-7, SKBr-3 and MDA-MB-231.

Although all-trans-retinoic acid (1) is a highly potent agonist for the RAR, its instability has led to a search for stable analogs (retinoids). 6-(5,6,7,8-tetrahydro-5,5,8,8-tetramethyl-2-naphthalenyl)-2-naphthalene carboxylic acid (TTNN, 2) is an RARP/γ selective agonist which has been shown to be active against ovarian, cervical and squamous cell carcinomas.^{[28-30]} As with other known RAR agonists, a major component of TTNN binding to RARs is via a hydrogen bonding network with the carboxylic acid.

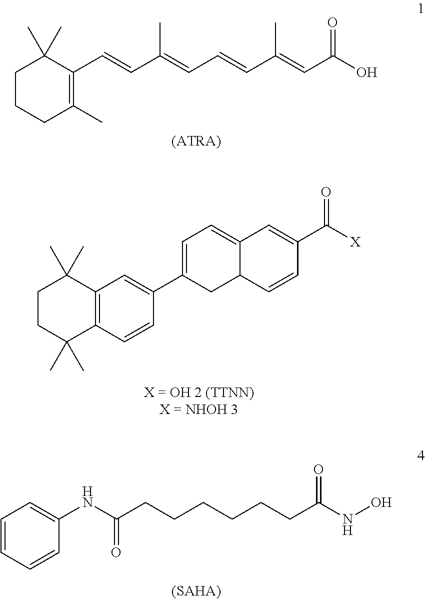

The isosteric replacement of the carboxylic acid in RAR agonists has been found to be very difficult, with standard carboxylic acids isosteres including sulfonic acids, sulfonamides, amidines and tetrazoles being unsuccessful when incorporated into retinoids and/or retinoic acid itself.^{[31]} A few exotic isosteres such as thiazolidindiones, 1,2,4-oxadiaxo1-5-ones and tropalones have been reported, but all result in molecules which bind to RAR less tightly and/or are less effective in inducing HL-60 differentiation when compared to ATRA and their parent retinoids.^{[32]}

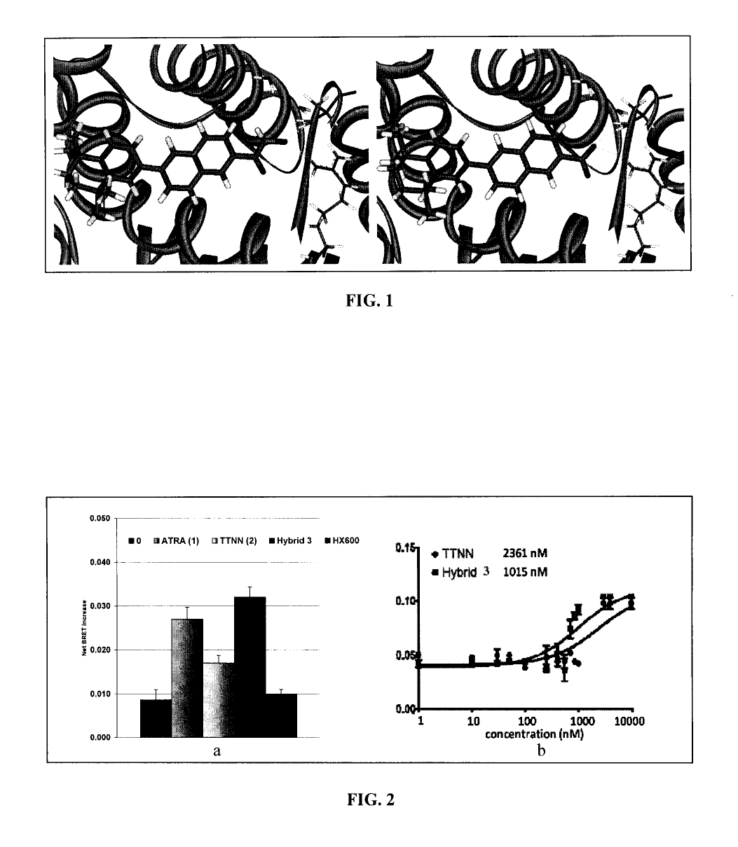

Despite difficulties in the use of carboxylic acid isosteres, it was surmised that a hydroxamic acid derivative of TTNN (e.g. 3) might be similar enough to enter into the required hydrogen bonding network in the receptor ligand binding pocket, allowing it to function as an RAR agonist. The binding of 3 with RAR using FITTED, a modeling suite for virtual screening, was investigated. FITTED reproduces binding modes of ATRA and other retinoids to RAR with high fidelity. Docking of 3 revealed that the hydroxamic acid can engage the same hydrogen bonding network as ATRA, TTNN and other retinoids, with the hydroxamate OH engaging a guanidine and serine in critical hydrogen bonds (FIG. 1). It was also surmised that the introduction of a hydroxamic acid into TTNN would potentially render the resulting molecule an HDACi while preserving its ability to act as an agonist for RARs.

Hybrid 3 was prepared by treatment of the methyl ester of TTNN with excess hydroxylamine under alkaline conditions. The effects of hybrid 3 on RAR were initially assessed using a bioluminescence resonance energy transfer (BRET) assay. The BRET assay monitors agonist-dependent recruitment of coactivators to RARs in transfected HEK293 cells, chosen because of their high degree of transfectability. Energy transfer between luciferase (fused to RARα) and eGFP (fused to the LXXLL coactivator motif) occurs only when these moieties are juxtaposed by the RAR-LXXLL interaction, and can be detected by the emission of green fluorescence. Assaying at 1 μM concentration, ATRA (1), TTNN (2) and hybrid 3 were all shown to be agonists of RARα while HX600, an RXR selective ligand, afforded no increase in BRET signal (FIG. 2a). Further dose-response comparison showed that 3 was in fact slightly more potent than 2 in inducing RARα (IC50=1.015 μM) (FIG. 2b).

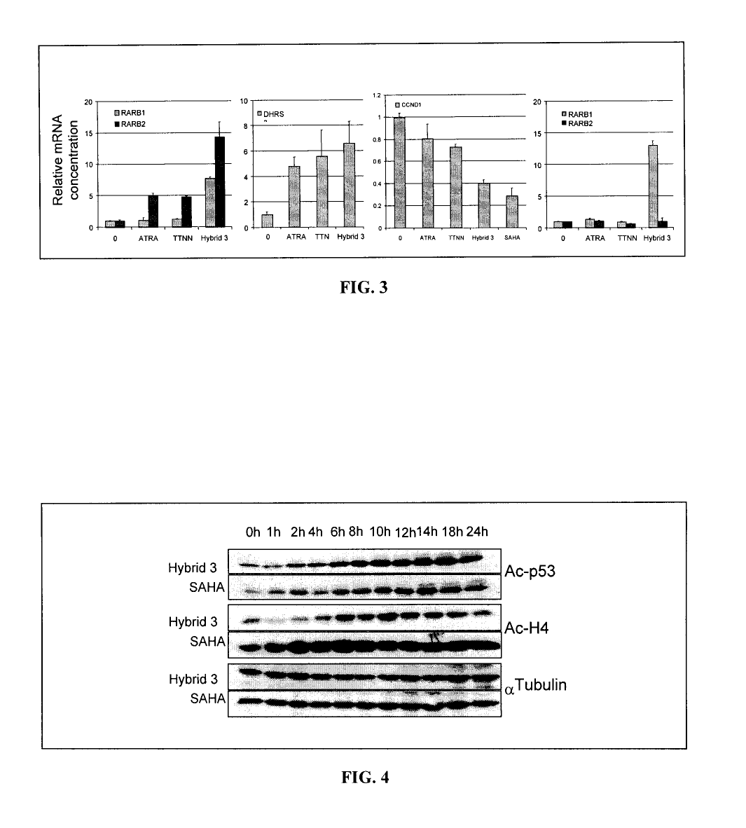

The agonist activity of 3 through its effects on RAR target genes was subsequently analyzed (FIG. 3). In both the MCF-7 and MDA-MB-231 cell lines, RT-qPCR analysis showed clear induction of RARB2 and DHRS3. In contrast, both 1 and 2 only induce RARB2 in the MDA-MB-231 cell line. In the MCF-7 cell line no induction of either RARB1 or RARB2 was observed. Furthermore, genes that are reportedly regulated by HDACi such as RARB1 and CyclinD1 follow similar regulation by 3 in MDA-MB-231.^{[33]}

The potential of 3 to act as an HDACi and cause deacetylation of a synthetic substrate or hyperacetylation of HDACs target proteins was subsequently assessed. Using a standard fluorescence assay, 3 was found to have IC50's of 5.0 μM and 148 nM against purified human HDAC3 and HDAC6, respectively. These potencies compare favorably to those observed with the vitamin D receptor agonist/HDACi hybrids against the same targets. Intracellular HDACi activity was assessed by measuring levels of acetylated histone H4 and tumor suppressor protein p53 in MDA-MB-231 cells by Western blotting. Similar to the effects observed with the highly potent HDACi SAHA, treatment with 10 μM 3 induced clear hyperacetylation of both HDAC targets, histone H4 and p53 over an 18-24 h time course (FIG. 4). Interestingly, hyperacetylation of p53 has been shown to cause its activation and the transcription of p53 target genes such as the pro-apoptotic genes PUMA, NOXA and BAX.

Finally, the effects of 3 on the growth of several breast cancer cell lines [MCF-7 (ER-positive, HER2-negative, RA-sensitive); SkBr3 (ER-negative, HER2-amplified, RA-sensitive); and the normally RA-insensitive MDA-MB-231 (ER-negative, HER2-negative, RA-insensitive)] was assessed.

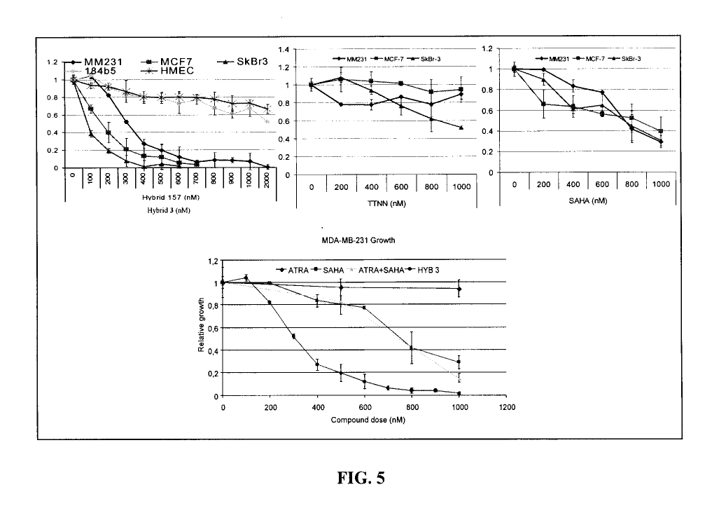

Impressively, under conditions where TTNN was inactive and SAHA had a shallow activity, hybrid 3 was active against all three cell lines, including MDA-MB231 (FIG. 5). IC50's of 300 nM, 150 nM and 90 nM against the MDA-MB-231, MCF-7 and SkBr3 cell lines were measured respectively. Hybrid 3 was also active against another ER-negative, HER2-negative and RA-insensitive cell line (BT-20). Importantly, 3 displayed only minimal effects on the growth of non-tumorogenic immortalized mammary 184b5 cells and normal human mammary epithelial cells (HMEC) as compared to SAHA which causes strong inhibition of HMEC growth, indicating a potentially useful therapeutic window. For comparison, in the MDA-MB-231 cell line, retinoids such as ATRA and TTNN had little to no effect, while treatment with SAHA induced a modest decrease in cell growth. Importantly, combination treatment of SAHA and ATRA did not potentiate either compound's activity and resulted in an inhibition very similar to that of SAHA, indicating an advantage of the hybrid molecule over combination therapy.

Hybrid 3 was shown to possess HDACi activity while maintaining RAR agonist activity. This hybrid molecule inhibits the growth of breast cancer cell lines that are both sensitive (MCF-7, SkBr-3) and resistant (MDA-MB-231, BT-20) to retinoids and is more efficient in these cells than the combination of retinoids with SAHA. Hybrid 3 was also shown to be less toxic than SAHA in normal and immortalized non-tumorogenic cell lines.

In an embodiment, the present specification relates to pharmaceutical compositions comprising a pharmaceutically effective amount of 3 or pharmaceutically acceptable salts thereof, in association with one or more pharmaceutically acceptable carriers, excipients and/or diluents. The term “pharmaceutically effective amount” is understood as being an amount of 3 required upon administration to a mammal in order to induce RAR agonism and HDAC inhibition. Therapeutic methods comprise the step of treating patients in a pharmaceutically acceptable manner with 3 or compositions comprising 3 as disclosed herein. Such compositions may be in the form of tablets, capsules, caplets, powders, granules, lozenges, suppositories, reconstitutable powders, creams, ointments, lotions, or liquid preparations, such as oral or sterile parenteral solutions or suspensions, or inhalation powders or solutions.

Hybrid 3 may be administered alone or in combination with pharmaceutically acceptable carriers. The proportion of each carrier is determined by the solubility and chemical nature of the agent(s), the route of administration, and standard pharmaceutical practice. In order to ensure consistency of administration, in an embodiment of the present disclosure, the pharmaceutical composition is in the form of a unit dose. The unit dose presentation forms for oral administration may be tablets and capsules and may contain conventional excipients. Non-limiting examples of conventional excipients include binding agents such as acacia, gelatin, sorbitol, or polyvinylpyrolidone; fillers such as lactose, sugar, maize-starch, calcium phosphate, sorbitol or glycine; tabletting lubricants such as magnesium stearate; disintegrants such as starch, polyvinylpyrrolidone, sodium starch glycolate or microcrystalline cellulose; or pharmaceutically acceptable wetting agents such as sodium lauryl sulphate. Additional excipients include those used in lipid formulations, a non-limiting example of which is olive oil.

Hybrid 3 may be administered alone or in combination with other pharmaceutically active molecules, including but not limited to cytotoxic, antiproliferative, pro-apoptotic and anti-inflammatory agents.

Hybrid 3 may be injected parenterally; this being intramuscularly, intravenously, or subcutaneously. For parenteral administration, 3 may be used in the form of sterile solutions containing solutes, for example sufficient saline or glucose to make the solution isotonic.

Hybrid 3 may be administered orally in the form of tablets, capsules, or granules, containing suitable excipients such as starch, lactose, white sugar and the like. Hybrid 3 may be administered orally in the form of solutions which may contain coloring and/or flavoring agents. Hybrid 3 may also be administered sublingually in the form of tracheas or lozenges in which the active ingredient(s) is/are mixed with sugar or corn syrups, flavoring agents and dyes, and then dehydrated sufficiently to make the mixture suitable for pressing into solid form.

The solid oral compositions may be prepared by conventional methods of blending, filling, tabletting, or the like. Repeated blending operations may be used to distribute the active agent(s) (i.e. hybrid 3) throughout the compositions, employing large quantities of fillers. Such operations are, of course, conventional in the art. The tablets may be coated according to methods well known in normal pharmaceutical practice, in particular with an enteric coating.

Oral liquid preparations may be in the form of emulsions, syrups, or elixirs, or may be presented as a dry product for reconstitution with water or any other suitable vehicle before use. Such liquid preparations may or may not contain conventional additives. Non limiting examples of conventional additives include suspending agents such as sorbitol, syrup, methyl cellulose, gelatin, hydroxyethylcellulose, carboxymethylcellulose, aluminum stearate gel, or hydrogenated edible fats; emulsifying agents such as sorbitan monooleate or acaci; non-aqueous vehicles (which may include edible oils), such as almond oil, fractionated coconut oil, oily esters selected from the group consisting of glycerine, propylene glycol, ethylene glycol, and ethyl alcohol; preservatives such as for instance methyl para-hydroxybenzoate, ethyl para-hydroxybenzoate, n-propyl para-hydroxybenzoate, n-butyl para-hydroxybenzoate and sorbic acid; and, if desired, conventional flavoring or coloring agents.

For parenteral administration, fluid unit dosage forms may be prepared by utilizing hybrid 3 and a sterile vehicle, and, depending on the concentration employed, hybrid 3 may be either suspended or dissolved in the vehicle. Once in solution, hybrid 3 may be injected and filter sterilized before filling a suitable vial or ampoule followed by subsequently sealing the carrier or storage package. Adjuvants, such as a local anesthetic, a preservative or a buffering agent, may be dissolved in the vehicle prior to use. Stability of the pharmaceutical composition may be enhanced by freezing the composition after filling the vial and removing the water under vacuum, (e.g., freeze drying). Parenteral suspensions may be prepared in substantially the same manner, except that hybrid 3 should be suspended in the vehicle rather than being dissolved and, further, sterilization is not achievable by filtration. Hybrid 3 may be sterilized, however, by exposing it to ethylene oxide before suspending it in the sterile vehicle. A surfactant or wetting solution may be advantageously included in the composition to facilitate uniform distribution of hybrid 3.

Topical administration can be used as the route of administration when local delivery of hybrid 3 is desired at, or immediately adjacent to the point of application of the composition or formulation comprising hybrid 3.

Hybrid 3 may also be dispensed as a dry or liquid inhalation formulation using any suitable device.

The pharmaceutical compositions of the present disclosure comprise a pharmaceutically effective amount of hybrid 3 as described herein and one or more pharmaceutically acceptable carriers, excipients and/or diluents. In an embodiment of the present disclosure, the pharmaceutical compositions contain from about 0.1% to about 99% by weight of hybrid 3. In a further embodiment of the present disclosure, the pharmaceutical compositions contain from about 10% to about 60% by weight of hybrid 3, depending on which method of administration is employed. Physicians will determine the most-suitable dosage of the present therapeutic agent (i.e. hybrid 3). Dosages may vary with the mode of administration of hybrid 3. In addition, the dosage may vary with the particular patient under treatment. The dosage of hybrid 3 used in the treatment may vary, depending on the condition, the weight of the patient, the relative efficacy and the judgment of the treating physician.

Both RARs and HDACs are ubiquitously expressed in cell lines and tissue. It was therefore surmised that hybrid 3 could display cytotoxic activities in a wide variety of cell lines. When tested in the NCI-60 panel of human tumor cell lines, hybrid 3 exhibited a broad spectrum cytotoxic activity across all tumor types. Under conditions where all cell lines show robust growth in the absence of test compound, treatment with hybrid 3 resulted in growth inhibition in all cell lines with GC-50 values ranging from 3.02×10⁻⁷M to 4.3×10⁻⁶M and total growth inhibition (TGI) concentrations ranging from 1.03×10⁻⁶M to 1.95×10⁻⁵ M. (Table 1). In addition, a measure of the loss of total protein content from time zero (TZ) shows that, with the exception of two Leukemia cell lines (CCRF-CEM and RPMI-8226), hybrid 3 exerts a cytotoxic effect in all cell lines with LC-50 ranging from 4.31×10^{×6}M to 4.65×10⁻⁵M.

TABLE 1
Functional studies of hybrid 3 on various cancer cell lines.

| Panel/Cell line | GI50 | TGI | LC50 |
|---|---|---|---|
| Leukemia | | | |
| CCRF-CEM | 3.06E−07 | 1.95E−06 | >0.0001 |
| HL-60(TB) | 3.53E−07 | 1.52E−06 | 7.66E−06 |
| K-562 | 3.99E−07 | 1.46E−06 | 5.51E−06 |
| MOLT-4 | 3.71E−07 | 1.46E−06 | 7.80E−06 |
| RPMI-8226 | 6.89E−07 | 4.39E−06 | >0.0001 |
| SR | 3.35E−07 | 1.20E−06 | 7.64E−06 |
| Non-Small cell | | | |
| lung cancer | | | |
| A549/ATCC | 4.73E−07 | 2.15E−06 | 7.80E−06 |
| EKVX | 6.86E−07 | 3.43E−06 | 1.84E−05 |
| HOP-62 | 6.16E−07 | 2.19E−06 | 6.18E−06 |
| HOP-92 | 1.93E−06 | 6.35E−06 | 2.72E−05 |
| NCI-H226 | 1.22E−06 | 4.19E−06 | 1.88E−05 |
| NCI-H23 | 9.96E−07 | 2.43E−06 | 5.90E−06 |
| NCI-H322M | 4.46E−07 | 3.50E−06 | 2.62E−05 |
| NCI-H460 | 3.40E−07 | 1.07E−06 | 4.04E−06 |
| NCI-H522 | 1.62E−06 | 3.59E−06 | 7.98E−06 |
| Colon | | | |
| COLO205 | 7.97E−07 | 2.33E−06 | 6.02E−06 |
| HCC-2998 | 1.09E−06 | 2.51E−06 | 5.75E−06 |
| HCT-116 | 4.24E−07 | 1.50E−06 | 4.03E−06 |
| HCT-15 | 3.81E−07 | 2.03E−06 | 9.38E−06 |
| HT29 | 5.75E−07 | 2.34E−06 | 7.60E−06 |
| KM12 | 6.16E−07 | 1.88E−06 | 4.61E−06 |
| SW-620 | 6.26E−07 | 1.94E−06 | 4.85E−06 |
| CNS | | | |
| SF-268 | 5.33E−07 | 1.85E−06 | 5.05E−06 |
| SF-295 | 3.85E−07 | 1.77E−06 | 6.11E−06 |
| SF-539 | 6.99E−07 | 7.26E−06 | 3.09E−05 |
| SNB-19 | 1.09E−06 | 2.36E−06 | 5.09E−06 |
| SNB-75 | 1.74E−06 | 6.10E−06 | 2.49E−05 |
| U251 | 4.37E−07 | 1.69E−06 | 4.80E−06 |
| Melanoma | | | |
| LOX IMVI | 3.72E−07 | 1.36E−06 | 4.19E−06 |
| M14 | 7.08E−07 | 2.49E−06 | 7.43E−06 |
| MDA-MB-435 | 8.79E−07 | 2.54E−06 | 6.80E−06 |
| SK-MEL-2 | 2.42E−06 | 6.31E−06 | 3.36E−05 |
| SK-MEL-28 | 1.81E−06 | 5.50E−06 | 2.17E−05 |
| SK-MEL-5 | 9.77E−07 | 2.31E−06 | 5.37E−06 |
| UACC-257 | 1.98E−06 | 7.34E−06 | 2.97E−05 |
| UACC-62 | 5.12E−07 | 1.86E−06 | 5.17E−06 |
| OVCAR-3 | 6.03E−07 | 1.88E−06 | 4.48E−06 |
| OVCAR-4 | 5.33E−07 | 5.82E−06 | 2.91E−05 |
| OVCAR-5 | 4.12E−06 | 1.95E−05 | 4.61E−05 |
| OVCAR-8 | 4.59E−07 | 1.77E−06 | 5.12E−06 |
| NCI/ADR-RES | 5.02E−07 | 1.76E−06 | 5.34E−06 |
| SK-OV-3 | 1.54E−06 | 1.17E−05 | 3.48E−05 |
| Renal | | | |
| 786-0 | 1.10E−06 | 2.37E−06 | 5.12E−06 |
| A498 | 1.66E−06 | 3.80E−06 | 8.71E−06 |
| ACHN | 3.93E−07 | 1.49E−06 | 5.09E−06 |
| CAKI-1 | 3.02E−07 | 1.03E−06 | 4.31E−06 |
| RXF393 | 1.26E−06 | 2.82E−06 | 6.27E−06 |
| SN12C | 1.05E−06 | 2.37E−06 | 5.34E−06 |
| TK-10 | 1.10E−06 | 4.97E−06 | 2.50E−05 |
| UO-31 | 4.04E−07 | 1.61E−06 | 4.53E−06 |
| Prostate | | | |
| PC-3 | 1.14E−06 | 4.25E−06 | 2.15E−05 |
| DU-145 | 6.22E−07 | 1.90E−06 | 4.68E−06 |
| Breast | | | |
| MCF7 | 4.70E−07 | 1.68E−06 | 4.66E−06 |
| MDA-MB-231/ATCC | 1.20E−06 | 3.59E−06 | 1.20E−05 |
| HS578T | 3.02E−06 | 1.18E−05 | 6.48E−05 |
| BT-549 | 1.42E−06 | 4.81E−06 | 1.97E−05 |
| T-47D | 1.27E−06 | 4.03E−06 | 2.80E−05 |
| MDA-MB-468 | 1.18E−06 | 2.75E−06 | 6.39E−06 |

Materials and Methods

Cell transfection and BRET assays: HEK293 cells were grown to confluence, trypsinized and plated at a density of 500 k cells per well (12-well plates) in DMEM supplemented with 10% charcoal treated FBS (FBS-T). The following day, cells were transfected with PEI using 0.1 μg of RAR-RLuc vector and 1 μg of LXXLL-eGFP vector. 48 hours post-transfection, cells were treated with retinoids for 2 hours before taking BRET measurements as described previously.

Western Blotting: MDA-MB-231 cells were plated in DMEM 5% FBS-T at 600 k cells per well in 6-well plates. The next day cells were treated with DMSO, TTNN (10 μM), Hybrid 3 (10 μM) or SAHA (150 nM) and the cells were collected in 95° C. Laemmli buffer at various time points. Protein concentration of the extracts was analyzed by BioRad DC protein assay. 20 μg of proteins per condition were loaded on an SDS-PA gel and transferred to a nitrocellulose membrane for blotting. Blotting was done using ABCAM primary antibodies EP356 (anti-p53) T3526 (anti-Tubulin) and AB1761 (anti-acH4).

Cell Growth Measurement: Cells were plated at 40 k cells per well (6-well plate) in DMEM 5% FBS-T for all cell lines except for I-IMEC cells which were plated in supplemented MEBM from Clonetics. Cells were treated every 48 hours and media was refreshed every 96 hours. After 10 days of treatment cells were harvested in 0.1N NaOH and growth was quantified by analyzing protein content of lysates with a Lowry assay.

In Vitro Cancer Screen

The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO₂, 95% air and 100% relative humidity for 24 h prior to addition of experimental drugs.

After 24 h, two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or ½ log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37° C., 5% CO₂, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and the plates are incubated for 10 minutes at room temperature. After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the absorbance measurements [time zero, (Tz), control growth, (C), and test growth in the presence of drug at the five concentration levels (Ti)], the percentage growth is calculated at each of the drug concentration levels. Percentage growth inhibition is calculated as: [(Ti-Tz)/(C-Tz)]×100 for concentrations for which Ti>/=Tz; and [(Ti-Tz)/Tz]×100 for concentrations for which Ti<Tz.

Three dose response parameters are calculated for each experimental agent. Growth inhibition of 50% (GI50) is calculated from [(Ti-Tz)/(C-Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz. The LC50 (concentration of drug resulting in a 50% reduction in the measured protein at the end of the drug treatment as compared to that at the beginning) indicating a net loss of cells following treatment is calculated from [(Ti-Tz)/Tz]×100=−50. Values are calculated for each of these three parameters if the level of activity is reached; however, if the effect is not reached or is exceeded, the value for that parameter is expressed as greater or less than the maximum or minimum concentration tested (Table 1).

It is to be understood that the disclosure is not limited in its application to the details of construction and parts as described hereinabove. The disclosure is capable of other embodiments and of being practiced in various ways. It is also understood that the phraseology or terminology used herein is for the purpose of description and not limitation. Hence, although the present disclosure has been provided with illustrative embodiments, it can be modified without departing from the spirit, scope and nature as further defined in the appended claims.

### REFERENCES

1. Wilcken N R, Sarcevic B, Musgrove E A, et al: Differential effects of retinoids and antiestrogens on cell cycle progression and cell cycle regulatory genes in human breast cancer cells. Cell Growth Differ 7:65-74, 1996.

2. Simeone A M, Tani A M: How retinoids regulate breast cancer cell proliferation and apoptosis. Cell Mol Life Sci 61:1475-84, 2004.

3. Mangiarotti R, Danova M, Alberici R, et al: All-trans retinoic acid (ATRA)-induced apoptosis is preceded by G1 arrest in human MCF-7 breast cancer cells. Br J Cancer 77:186-91, 1998.

4. Toma S, Isnardi L, Raffo P, et al: Effects of all-trans-retinoic acid and 13-cis-retinoic acid on breast-cancer cell lines: growth inhibition and apoptosis induction. Int. J Cancer 70:619-27, 1997.

5. Toma S, Isnardi L, Riccardi L, et al: Induction of apoptosis in MCF-7 breast carcinoma cell line by RAR and RXR selective retinoids. Anticancer Res 18:935-42, 1998.

6. Toma S, Isnardi L, Raffo P, et al: RARalpha antagonist Ro 41-5253 inhibits proliferation and induces apoptosis in breast-cancer cell lines. Int. J Cancer 78:86-94, 1998.

7. Freemantle S J, Spinella M J, Dmitrovsky E: Retinoids in cancer therapy and chemoprevention: promise meets resistance. Oncogene 22:7305-15, 2003.

8. Veronesi U, De Palo G, Marubini E, et al: Randomized trial of fenretinide to prevent second breast malignancy in women with early breast cancer. J Natl Cancer Inst 91:1847-56, 1999.

9. Zanardi S, Serrano D, Argusti A, et al: Clinical trials with retinoids for breast cancer chemoprevention. Endocr Relat. Cancer 13:51-68, 2006.

10. Lacroix A, L'Heureux N, Bhat PV: Cytoplasmic retinoic acid-binding protein in retinoic acid-resistant human breast cancer sublines. J Natl Cancer Inst 73:793-800, 1984.

11. Ueda H, Ono M, Hagino Y, et al: Isolation of retinoic acid-resistant clones from human breast cancer cell line MCF-7 with altered activity of cellular retinoic acid-binding protein. Cancer Res 45:3332-8, 1985.

12. Yang L, Kim H T, Munoz-Medellin D, et al: Induction of retinoid resistance in breast cancer cells by overexpression of cJun. Cancer Res 57:4652-61, 1997.

13. Stephen R, Darbre P D: Loss of growth inhibitory effects of retinoic acid in human breast cancer cells following long-term exposure to retinoic acid. Br J Cancer 83:1183-91, 2000.

14. Raffo P, Emionite L, Colucci L, Belmondo F, Moro M G, Bollag W, Toma S. Retinoid receptors: pathways of proliferation inhibition and apoptosis induction in breast cancer cell lines. Anticancer Res. 20(3A):1535-43, 2000.

15. van der Burg B, van der Leede B M, Kwakkenbos-Isbrucker L, et al: Retinoic acid resistance of estradiol-independent breast cancer cells coincides with diminished retinoic acid receptor function. Mol Cell Endocrinol 91:149-57, 1993.

16. Fitzgerald P, Teng M, Chandraratna R A, et al: Retinoic acid receptor alpha expression correlates with retinoid-induced growth inhibition of human breast cancer cells regardless of estrogen receptor status. Cancer Res 57:2642-50, 1997.

17. Sheikh M S, Shao Z M, Li X S, et al: Retinoid-resistant estrogen receptor-negative human breast carcinoma cells transfected with retinoic acid receptor-alpha acquire sensitivity to growth inhibition by retinoids. J Biol Chem 269:21440-7, 1994.

18. Rosenauer A, Nervi C, Davison K, et al: Estrogen receptor expression activates the transcriptional and growth-inhibitory response to retinoids without enhanced retinoic acid receptor alpha expression. Cancer Res 58:5110-6, 1998.

19. Rousseau C, Pettersson F, Couture M C, et al: The N-terminal of the estrogen receptor (ERalpha) mediates transcriptional cross-talk with the retinoic acid receptor in human breast cancer cells. J Steroid Biochem Mol Biol 86:1-14, 2003.

20. Rousseau C, Nichol J N, Pettersson F, et al: ERbeta sensitizes breast cancer cells to retinoic acid: evidence of transcriptional crosstalk. Mol Cancer Res 2:523-31, 2004.

21. Siwak D R, Mendoza-Gamboa E, Taxi A M: HER2/neu uses Akt to suppress retinoic acid response element binding activity in MDA-MB-453 breast cancer cells. Int. J Oncol 23:1739-45, 2003.

22. Rocha W, Sanchez R, Deschenes J, et al: Opposite effects of histone deacetylase inhibitors on glucocorticoid and estrogen signaling in human endometrial Ishikawa cells. Mol Pharmacol 68:1852-62, 2005.

23. Fuino L, Bali P, Wittmann S, et al: Histone deacetylase inhibitor LAQ824 down-regulates Her-2 and sensitizes human breast cancer cells to trastuzumab, taxotere, gemcitabine, and epothilone B. Mol Cancer Ther 2:971-84, 2003.

24. Bali P, Pranpat M, Swaby R, et al: Activity of suberoylanilide hydroxamic Acid against human breast cancer cells with amplification of her-2. Clin Cancer Res 11:6382-9, 2005.

25. Emionite L, Galmozzi F, Grattarola M, et al: Histone deacetylase inhibitors enhance retinoid response in human breast cancer cell lines. Anticancer Res 24:4019-24, 2004.

26. Yokota T, Matsuzaki Y, Miyazawa K, et al: Histone deacetylase inhibitors activate INK4d gene through Sp1 site in its promoter. Oncogene 23:5340-9, 2004.

27. Tavera-Mendoza L, Wang T T, Lallemant B, Zhang R, Nagai Y, Bourdeau V, Ramirez-Calderon M, Desbarats J, Mader S, White J H. Convergence of vitamin D and retinoic acid signaling at a common hormone response element. EMBO Rep. 2006, 7, 180-5.

28. Wan H, Oridate N, Lotan D, Hong W K, Lotan R. Overexpression of retinoic acid receptor beta in head and neck squamous cell carcinoma cells increases their sensitivity to retinoid-induced suppression of squamous differentiation by retinoids. Cancer Res. 1999 Jul 15; 59(14):3518-26.

29. Harant H, Korschineck I, Krupitza G, Fazeny B, Dittrich C, Grunt T W. Retinoic acid receptors in retinoid responsive ovarian cancer cell lines detected by polymerase chain reaction following reverse transcription. Br J Cancer 1993 Sep; 68(3):530-6.

30. Lotan R, Dawson M I, Zou C C, Jong L, Lotan D, Zou C P. Enhanced efficacy of combinations of retinoic acid- and retinoid X receptor-selective retinoids and alpha-interferon in inhibition of cervical carcinoma cell proliferation. Cancer Res. 1995 Jan 15; 55(2):232-6.

31. Yamakawa, T.; Kagechika, H.; Kawachi, E.; Hashimoto, Y.; Shudo, K. J. Med. Chem. 1990, 33, 1430. b) Dawson, M. I,; Hobbs, P. D.; Kuhlmann, K.; Fung, V. A.; Helmes, C. T.; Chao, W.-R. J. Med. Chem. 1980, 23, 1013.

32. a) Charton, J.; Deprez-Poulain, R.; Hennuyer, N.; Tailleux, A.; Staels, B.; Deprez, B. Bioorg. Med. Chem. Lett. 2009, 19, 489. b) Tashima, T.; Kagechika, H.; Tsuji, M.; Fukusawa, H.; Kawachi, E.; Hashimoto, Y.; Shudo, K. Chem. Pharm. Bull. 1997, 1805. c) Ebisawa, M.; Ohta, K.; Kawachi, E.; Fukusawa, H.; Hashimoto, Y.; Kagechika, H. Chem. Pharm. Bull. 2001, 49, 501.

33. De los Santos M, Zambrano A, Sánchez-Pacheco A, Aranda A. Histone deacetylase inhibitors regulate retinoic acid receptor beta expression in neuroblastoma cells by both transcriptional and posttranscriptional mechanisms. Mol Endocrinol. 21(10):2416-26, 2007.

1. A hybrid molecule comprising a retinoic acid receptor agonist moiety and a histone deacetylase (HDAC) inhibitory moiety.

2. The hybrid molecule of claim 1 or a pharmaceutically acceptable salt thereof having the formula:

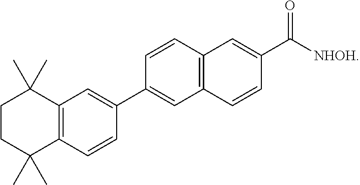

3. A composition comprising the hybrid molecule according to claim 2 and a pharmaceutically acceptable carrier or diluent.

4. A pharmaceutical composition comprising the hybrid molecule according to claim 2 or a pharmaceutically acceptable salt thereof and a pharmaceutically acceptable carrier.

5. The pharmaceutical composition of claim 4, wherein the composition is presented in a form selected from the group consisting of capsules, granules, powders, solutions, suspensions, and tablets.

6. The pharmaceutical composition of claim 5, wherein the composition is administered by a method selected from the group consisting of oral, sublingual, buccal, parenteral, intravenous, transdermal, inhalation, intranasal, vaginal, intramuscular, and rectal modes of administration.

7. A method of treating cancer in a subject, the method comprising administering to the subject a therapeutically effective amount of a hybrid molecule according to claim 2 or a pharmaceutically acceptable salt thereof, wherein the cancer is a breast cancer, a leukemia, a non-small cell lung cancer, a colon cancer, a melanoma, an ovarian cancer, a renal cancer, a prostate cancer, or a cancer of the central nervous system.

8.-15. (canceled)

16. The method of claim 7, wherein the subject is an in vitro or in vivo system.

17. The method of claim 7, wherein the subject is a human.
